(19) United States
(12) Patent Application Publication
(10) Pub. No.: US 2023/0149668 A1
(43) Pub. Date: May 18, 2023

(54) THREE-DIMENSIONAL PROTECTIVE DEVICE

(71) Applicant: Aidan Raney, Newport Beach, CA (US)

(72) Inventor: Aidan Raney, Newport Beach, CA (US)

(21) Appl. No.: 17/987,733

(22) Filed: Nov. 15, 2022

Related U.S. Application Data
(60) Provisional application No. 63/279,573, filed on Nov. 15, 2021.

(51) Int. Cl. A61M 25/09

(52) U.S. Cl. CPC A61M 25/09; A61M 2025/09175; A61M 2025/09083

(57) ABSTRACT

Disclosed are example embodiments of a guidewire. The guidewire includes an elongated core with a proximal end, a distal end, and a length extending along a longitudinal axis therebetween, and a three-dimensional atraumatic tip disposed at the distal end of the elongated core. The three-dimensional atraumatic tip may include a spherical shaped coil of wire. The three-dimensional atraumatic tip may include an oblong shaped coil of wire. The three-dimensional atraumatic tip may include an umbrella shaped coil of wire.

### CLAIM OF PRIORITY UNDER 35 U.S.C. § 119

The present Application for Patent claims priority to Provisional Application No. 63/279,573 entitled “THREE-DIMENSIONAL PROTECTIVE DEVICE” filed Nov. 15, 2021, and assigned to the assignee hereof and hereby expressly incorporated by reference herein.

### TECHNICAL FIELD

The disclosure relates generally to the field of medical procedures, specifically and not by way of limitation; some embodiments are related to devices used for medical procedures.

### BACKGROUND

Valvular heart disease (VHD) is a common cause of morbidity and mortality, particularly among elderly patients. Aortic stenosis and degenerative mitral regurgitation are the most common valvular heart diseases in the world. With an aging population across the globe, the prevalence of these and other valvular diseases are expected to grow considerably. Increasingly, percutaneous techniques and transcatheter devices are being used to treat patients with various forms of VHD in place of open surgical approaches given the reduced morbidity and comparable outcomes of these minimally invasive approaches.

Transcatheter aortic valve replacement (TAVR) and transcatheter mitral valve replacement (TMVR) are two percutaneous techniques utilized to treat patients with aortic and mitral valvular disease, respectively. TAVR is an FDA approved therapy for severe, symptomatic aortic stenosis in native or bioprosthetic valves that has now been performed in over 250,000 patients each year and has surpassed the annual number of surgical aortic valve replacements in the United States. TMVR is an emerging percutaneous therapy that is anticipated to have exponential growth in the coming years as clinical data supports the efficacy and safety of the TMVR approach.

TAVR and TMVR are examples of medical procedures that use devices that are advanced over a guidewire. The guidewire may be placed in the left ventricle and used as a guide or rail to advance the device into the desired location. In some cases, the guidewire may lead to injury of the left ventricle. The injury of the left ventricle is known as left ventricular perforation. Left ventricular perforation is the leading cause for emergency cardiac surgery, and mortality from emergent cardiac surgery secondary to left ventricular perforation has been reported at 46% in a large registry study.

FIG. 19 is a diagram illustrating an example 2D guidewire. The 2D guidewire may include a spiral end. The spiral end of the 2D guidewire may provide some protection against perforating the heart. However, the spiral end of the 2D guidewire may not distribute stress and force from the guidewire enough to avoid perforating the left ventricle. Accordingly, a guidewire that is even less likely to perforate the heart, e.g., less likely to perforate the left ventricle is needed.

### SUMMARY

Disclosed are systems and methods for a 3-dimensional (3D) shape guidewire. The 3D guidewire proposed may better distribute stress and force from the wire to reduce the risk of damage to cardiac structures during the delivery of intravascular and endoscopic procedures. In one example, the primary use case demonstrated here describes a guidewire, which is the general mechanism for delivering cardiac devices into the body and desired location. As described in the figures below, the 3-dimensional device may be used in similar form to protect surrounding bodily structures during the delivery of intravascular and endoscopic procedures.

An example guidewire is disclosed. The example guidewire may include an elongated core with a proximal end, a distal end, and a length extending along a longitudinal axis therebetween, and a three-dimensional atraumatic tip disposed at the distal end of the elongated core.

A method of a guidewire is disclosed. The method may include providing the guidewire, the guidewire including an elongated core with a proximal end, a distal end, and a length extending along a longitudinal axis therebetween and a three-dimensional atraumatic tip disposed at the distal end of the elongated core, at least one of the elongated core and the atraumatic tip disposed within a sheath prior to deployment the elongated core and the atraumatic tip. The method including deploying the elongated core and the atraumatic tip.

The features and advantages described in the specification are not all-inclusive, and in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the disclosed subject matter.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 is a diagram illustrating an example 3D guidewire in accordance with the systems and methods described herein.

FIG. 2 is a diagram further illustrating the example 3D guidewire of FIG. 1.

FIG. 3 is a diagram illustrating the example 3D guidewire of FIGS. 1 and 2 exiting a sheath.

FIG. 4 is a diagram illustrating an example 3D guidewire including a junction point.

FIG. 5 is a diagram illustrating an example 3D guidewire including a standard guidewire and a guidewire in accordance with the systems and methods described herein.

FIG. 6 is a diagram illustrating an example 3D guidewire including a standard J wire and a guidewire in accordance with the systems and methods described herein.

FIG. 7 is a diagram illustrating an example 3D guidewire including a standard J wire and a guidewire in accordance with the systems and methods described herein.

FIG. 8 is a diagram illustrating an example device when expanded in accordance with the systems and methods described herein.

FIG. 9 is a diagram illustrating an example device when expanded in accordance with the systems and methods described herein.

FIG. 10 is a diagram illustrating an example side view of the device including a small wire inside and outer balloon that may expand the device when the balloon is expanded in accordance with the systems and methods described here.

FIG. 11 is a diagram illustrating an example device that may be placed around an existing guidewire and inserted into a body in accordance with the systems and methods described herein.

FIG. 12 is a diagram illustrating an example device that may be placed around an existing guidewire and inserted into a body in accordance with the systems and methods described herein.

FIG. 13 is a diagram illustrating an example device inserted into a body in accordance with the systems and methods described herein.

FIG. 14 is a diagram illustrating an example device inserted into a body in accordance with the systems and methods described herein.

FIGS. 15A-15B are diagrams demonstrating a 3-dimensional view of an example embodiment of the proposed device.

FIGS. 16A-16B are diagrams demonstrating use of an example device on a cannula.

FIGS. 17A-17B are diagrams demonstrating use of an example device with transesophageal echocardiography (TEE).

FIG. 18 is a diagram illustrating how various devices, such as cardiac devices may advance into the body over standard sized guidewires.

FIG. 19 is a diagram illustrating an example 2D guidewire.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

The detailed description set forth below in connection with the appended drawings is intended as a description of configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well-known structures and components are shown in block diagram form in order to avoid obscuring such concepts. Several aspects of guidewires will now be presented with reference to various apparatus and methods.

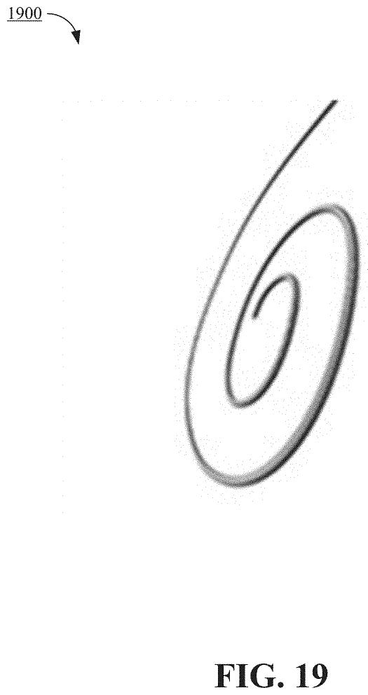

As discussed above with respect to FIG. 19 guidewires have been developed with pre-curved shapes, e.g., a spiral end, to distribute some of the forces imparted to the wire. An example is the Safari 2 wire 1900 illustrated in FIG. 19. The pre-curved shape distributes some force, but the force is still along a 2D line. Distributing force along a 2D line may still lead to cardiac damage.

As used herein, a “2D” shape for a guidewire refers to a guidewire that generally has a planar shape. In an informal sense, the 2D guidewire may lie within a plane. Informal because a geometric plane technically has no thickness. The guidewire is a real-world device, however, is not an abstract geometric construct. Accordingly, the “2D” guidewire has some thickness. For example, the spiral of FIG. 19 is generally flat or level. This is not to say that the spiral has no thickness, but rather that the spiral generally has a thickness at or near the thickness of the wire used to form the guidewire.

There is currently no pre-shaped wire that takes on a 3-dimensional (3D) structure in the heart. The systems and methods described herein propose a wire that takes on a 3D shape to distribute forces more evenly on the heart and other vascular structures. The 3D shape may provide a greater surface area to absorb forces imparted to the guidewire should the guide wire 3D structure contact the heart. For example, when the 3D structure contacts the heart, it may make contact in multiple locations rather than at an end of a wire or along a 2D structure.

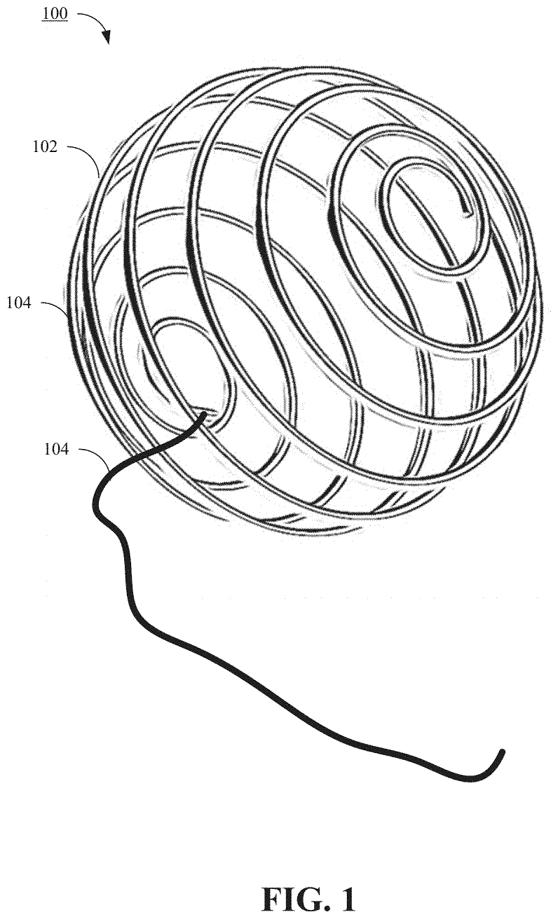

FIG. 1 is a diagram illustrating an example 3D guidewire 100. Disclosed are systems and methods for a 3D shape guidewire 100. The 3D guidewire 100 proposed may better distribute stress and force from the wire to reduce the risk of damage to cardiac structures. As used herein a 3D guidewire 100 may refer to a guidewire that is generally not a planar shape. The 3D guidewire 100 may include portions that extend outside a plane. The 3D guidewire 100 may generally include a coil like structure 102 that forms a structure in three dimensions that is thicker than the thickness of the wire 104 itself. For example, the spherically shaped coil (e.g., coil like structure 102) of FIG. 1 is one example of a 3D guidewire. It will be understood that this is only one embodiment. A 3D guidewire does not have to be spherically shaped. For example, other embodiments may be oblong. For example, a 3D guidewire may be elongated from the spherically shape of the 3D shape guidewire 100. In some embodiments, the shape may generally be curved. A curved shape, particularly a 3D curved shape may better distribute forces from the guidewire to better avoid or hopefully better avoid cardiac damage.

Cardiac devices may be advanced into the body over standard sized guidewires. For example, a transcatheter aortic valve may be advanced over a 0.035-inch guidewire. These wires may be advanced through a catheter that has an inner diameter just larger than the standard guidewire. As the wire is advanced through the delivery catheter, it may take a shape along an X and Y axis. In an example embodiment, a proposed device may be designed such that the example embodiment may be delivered through a standard catheter, but take a geometric spherical form along an X, Y, and Z axis. Other embodiments may take other geometric shapes in the X, Y, and Z axis, e.g., other 3-dimensional shapes.

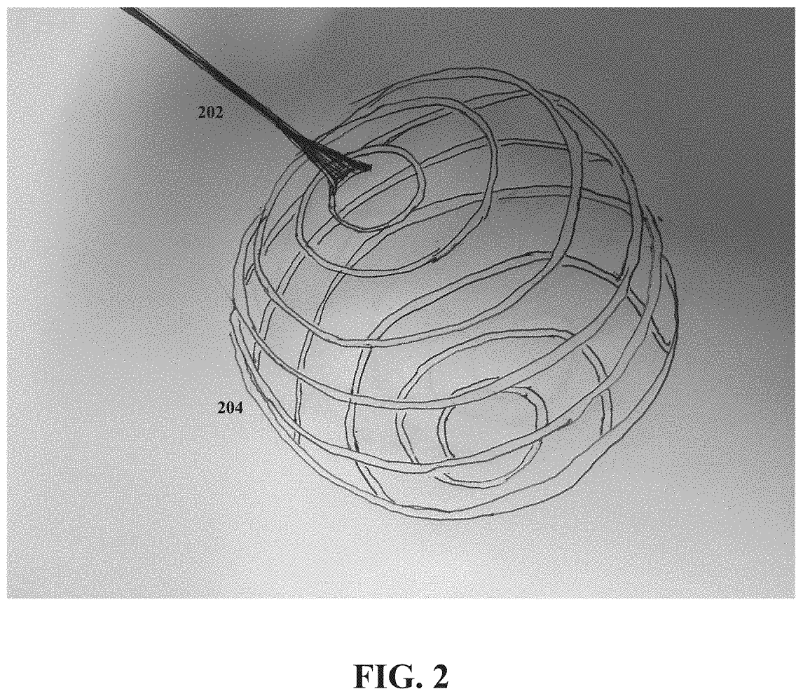

FIG. 2 is a diagram further illustrating the example 3D guidewire of FIG. 1. An example embodiment includes a device that may include two main elements. The two main elements may be a wire portion 202 and a 3D wire having a 3D shape 204, e.g., a sphere (204), an oblong shape, or other 3D geometric shapes.

An example embodiment may include a 3D shape, which may be designed using Nitinol. Nitinol may be a nickel-titanium alloy with super elasticity and shape memory properties. Shape memory may refer to the ability of the material, e.g., Nitinol, to undergo deformation and recover to the material's original shape. A shape-memory alloy (SMA) may be an alloy that can be deformed, e.g., when the alloy is cold but returns to its pre-deformed (“remembered”) shape, e.g., when the alloy is heated. Superelasticity may be an elastic (reversible) response to an applied stress, caused by a phase transformation between the austenitic and martensitic phases of a crystal. Superelasticity may be exhibited in shape-memory alloys.

An example embodiment may include a nitinol (or other metal, alloy) structure surrounded by a balloon that may be expanded to provide safety to surrounding structures. Another example embodiment may be a balloon expandable 3-dimensional device with no inner nitinol or other metal structure.

An example embodiment may be engineered such that the wire may conduct and sense electricity. For example, the guidewire may be made from materials that may ensure conduction of electricity. An example embodiment may be engineered such that the wire may conduct, and sense electricity. Use of a wire that may conduct and sense electricity may provide for an ability to be used as a set of leads for a temporary pacemaker. For example, a device may be connected to the guidewire causing electrical current to be delivered to surrounding heart structures in a unipolar fashion in order to provide a pacemaker function, e.g., electrical current to be delivered to surrounding heart structures in a fashion having or relating to a single pole or kind of polarity. The increased surface area of the end of the guidewire may increase delivery of electrical charge to the cardiac structure, e.g., due to the increased surface area.

An example embodiment may have an ability to sense pressures within the heart. The ability to sense pressures within the heart is known as hemodynamics. Hemodynamics within the heart relate to the pressures of the blood flow within the heart.

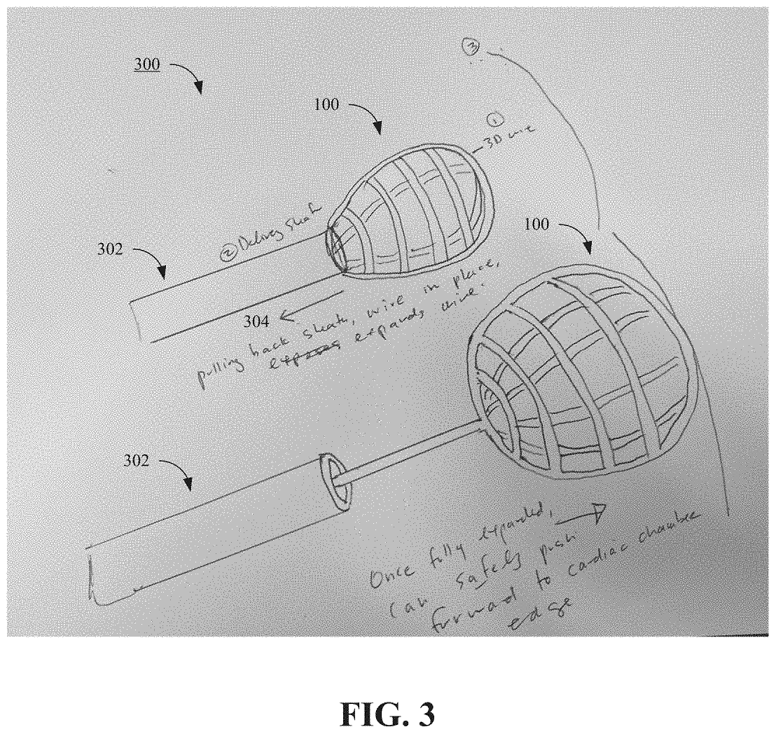

FIG. 3 is a diagram 300 illustrating the example 3D guidewire of FIGS. 1 and 2 exiting a sheath 302. The sheath 302 may form a delivery shaft. An example embodiment may have an ability to be pushed forward, e.g., the sheath 302 may be pushed forward within a blood vessel or GI tract and towards a chamber. The example embodiment may take proper form, e.g., a form to minimize damage to the surrounding structure (e.g., cardiac chamber).

Alternatively, in some embodiments, the 3D structure of the guidewire may be within a delivery sheath 302. The 3D structure may be collapsed within the sheath/delivery shaft, e.g., initially. For example, in some embodiments, the 3D structure of the guidewire may be collapsed within the delivery sheath 302. The delivery sheath 302 may simply be pulled backwards 304 to expose the guidewire 100. In some embodiments, the 3D structure of the guidewire 100 as well as a portion of the elongated wire portion of the guidewire may be within a delivery sheath 302. Pulling back the sheath 302 in the middle of a cardiac chamber to expose the 3D wire may minimize potential for cardiac damage. Furthermore, once the 3D structure is fully expanded, the 3D structure may generally be pushed forward to the cardiac chamber edge safely. The 3D structure may protect the heart from damage. For example, the 3D structure may spread any pressure imparted to the guidewire over a greater surface area of the heart walls.

In an example embodiment, once the 3D wire is in place and expanded, the 3D wire may safely be pushed into contact with the bodily structure (e.g., ventricle or the atrium if used in the heart). The 3D wire may spread the given force over a greater area, e.g., within the ventricle or the atrium of the heart.

In an example embodiment, once the 3D wire is in place, the 3D wire may have the ability to manipulate a junction point such that the supported device may move to a proper position within the valve, e.g., the value to the ventricle or the atrium of the heart.

In an example embodiment, once the 3D wire is in place, a device may be pushed forward on the wire at the same time to help position the valve.

A junction point may be engineered with an ability to move with a certain amount of force applied, e.g., a force dependent ratchet. In an example embodiment, a hinge point may move some angular distance, e.g., 5 degrees, to align a valve. Additionally, an example embodiment may have flexion at the hinge point. The force dependent ratchet may be a ratchet that may be set to a particular torque setting. The force dependent ratchet may enable the device being positioned to be better aligned with a valve or a cardiac structure in question. Pulling back into the sheath 302 or device delivery system may restore an original position of the junction point.

Currently, a surgeon may only push in a guide wire 100 or pull out the guide wire 100 in order to attempt to align a valve that is being added to a heart, e.g., as part of a TAVR medical procedure or a TMVR medical procedure. If considering anterior/posterior or superior/inferior, the surgeon may only be able to align the valve in one major axis, e.g., along the trajectory that the guide wire takes naturally within a plane, for example, along the trajectory of the guidewire with no ability to change the one major axis. Alignment of the valve within the native annulus plane in all directions may impact the function of the valve. When the valve is misaligned, there may be leakage around the valve, known as paravalvular leak.

The example wire may align “East-West,” for example. As used herein, “East-West” defines a direction in the plane of a native valve annulus, rather than a geographic compass direction. As a device is advanced across the native annulus, there may not be a way to control for alignment in direction perpendicular to the first axis. As used herein, “North-South” defines a direction perpendicular to the first axis but within the same plane. For example, a surgeon may be able to advance a device over a guidewire and align the East-West plane. The valve may be misaligned in the North-South plane. However, when a surgeon pushes on the wire and the valve at the same time the surgeon may align both East-West and North-South. Accordingly, the device may then be positioned such that the deployment may be predictable and reproducible. With current wires this is not a safe maneuver, as pushing on wire and valve at the same time may lead to a puncture in the corresponding vessel or chamber in the heart. In an example, using the systems and methods described herein, the 3-D wire may allow for this to be performed safely, e.g., the probability of puncturing the corresponding vessel or chamber in the heart may be decreased or eliminated relative to previous systems.

Flexion of the junction point, e.g., flexing or bending of the junction point, may give the surgeon the ability to manipulate the device being delivered in an additional direction. Accordingly, some adjustment of the device being delivered in two dimensions may be possible. Additionally, after flexion, the wire may be rotated clockwise or counterclockwise. Rotating the device being delivered clockwise or counterclockwise may give the additional dimension of alignment. Furthermore, while pushing on the wire and device at the same time may give the additional movement of alignment, the alignment movement may not be safe. The alignment movement may not be safe because the two-dimensional component of currently shaped wires may lead to a perforation. Such a heart perforation generally has a very high mortality rate. 3-dimensional wire may enable this to be done safely.

In an example of a valve in ring procedures, when one direction is off more than, e.g., 17 degrees, the covering skirt may not be in all directions, leading to paravalvular leak. A paravalvular leak is a potential complication of both mechanical and bioprosthetic valve implants, with an incidence of 2% to 10% in the aortic position and 7% to 17% in the mitral position. The paravalvular leak may have varying degrees of severity. However, the paravalvular leak may be clinically significant. The proposed device may mitigate paravalvular leak through the ability to more accurately align the valve during deployment.

As described herein, an example embodiment may include a delivery sheath 302, e.g., as illustrated in FIG. 3. An example embodiment may be used to deliver a guidewire. For example, the 3D structure may be at an end of the guidewire and may allow the guidewire to be more safely extended through a vessel to the heart. A portion of the delivery of the guidewire may be through a sheath.

An example embodiment may be advanced over previously placed wire. For example, the 3D structure and/or the guidewire of the 3D structure may be advanced over previously placed wire. In another example embodiment, a sheath may be advanced over previously placed wire. The prior wire may then be removed. The 3D structure may be advanced through the sheath that was placed over previously placed wire.

In an example, a standard wire is may be 0.035 inches for heart surgeries involving valves and 0.014 inches for surgeries involving the coronary artery. An example surgery may start with the standard wire, e.g., 0.035 inches thick. Once the standard wire is in place, the surgeon may advance an exchange catheter, also referred to as an exchange sheath along the standard wire. The surgeon may then have an open tube where the surgeon wants the wire to end up. The wire described herein may then be advanced through the tube.

Delivery and/or exchange of the sheath may have an inner dilator and an outer sheath. The outer sheath and the inner dilator may be advanced over the standard 0.035 guidewire. The inner dilator may then be removed, which creates a larger inner lumen. The 3D guidewire described herein may then be advanced through the larger inner lumen. In an example, the core wire may need to be 0.035 inches, for example, to deliver a standard transcatheter valve. The proposed wire may need a larger lumen to deliver the 3D component. The use of an inner dilator and an outer dilator may enable delivery of the proposed device over standard guidewires. Note that the device may be manufactured to all standard wire sizes, as different wire sizes are used for different applications in the body.

An example embodiment may have the ability to measure pressure. For example, one embodiment may use a small tube running along the core wire and connecting to a standard pressure sensor, e.g., on a table in the operating room. In another embodiment, the sheath itself may measure pressure. However, these are only examples, any appropriate pressure measuring device for use in surgery known or developed may be used.

As described herein, in an example embodiment a sheath may be pulled back. The example embodiment may allow the wire, e.g., the 3D end of the guidewire, to assume a full three-dimensional shape.

An example embodiment a sheath may be made from steel or other metal material. In another embodiment, the sheath may be made from other suitable materials other than metal. The sheath may be disposed around the core of the guidewire. In an example embodiment, the sheath may be configured to retract against the core of the guidewire. When the sheath is retracted against the core of the guidewire, the 3D structure may expand. When the 3D structure expands, the 3D structure may perform its heart protecting function of spreading forces over a greater surface area to protect the heart from a perforation.

In an example embodiment the guidewire and/or the 3D structure may be made from nitinol. A nitinol wire may be packaged in a manner that maintains final 3D shape. Accordingly, the 3D structure may be configured to maintain its 3D shape, e.g., after the 3D structure exits the sheath. Thus, the guidewire and 3D structure may be guided along a vessel until it at the desired position within the cardiac chamber. When the guidewire and 3D structure are at the desired location, the 3d structure could be pushed forward to expand, or expand by pulling back the delivery sheath. For example, in transcatheter valve replacement, the device is advanced from the femoral artery or vein and into the heart. In another example where the device is used within a coronary artery, the delivery sheath would be advanced over a pre-existing wire that is deep in the coronary artery. Once the sheath is advanced, the initial wire would be removed, and the 3D wire would be advanced through the delivery sheath into the desired position.

An example embodiment may include one or more radiopaque markers. Radiopaque markers may facilitate target vessel access and enable accurate positioning during procedures. In one example, a radiopaque marker may include an X-ray contrast agent, e.g., a liquid form that we inject to see structures. For example, the radiopaque marker may include a solid X-ray contrast agent. The X-ray contrast agent may show up on an X-ray. In other examples, the radiopaque marker may include a contrast agent that may be imaged using some other imaging technology, e.g., an imaging technology other than X-ray.

Example embodiments may be used with all heart valves, e.g., an example embodiment may be used with an aortic valve. Another example embodiment may be used with a mitral valve. An example embodiment may be used with a tricuspid valve. Another example embodiment may be used with a pulmonic valve. An example embodiment may be used to safely deliver other cardiac devices, e.g., a MitraClip or WATCHMAN. A MitraClip (mitral clip) may be a medical device that may be used to treat mitral valve regurgitation for individuals who should not have open-heart surgery. The MitralClip may be implanted via a tri-axial transcatheter technique, e.g., using the systems and methods described herein. Additionally, the MitralClip may involve suturing together the anterior and posterior mitral valve leaflets. The WATCHMAN Implant is a minimally invasive, one-time procedure designed to reduce the risk of strokes that originate in the left atrial appendage (LAA).

An example embodiment may provide a good support structure for paravalvular leak closure. In an example, a surgeon may insert a catheter into the femoral vein in the groin for paravalvular leak closure. A guidewire, e.g., including the systems and methods described herein, may be used to guide the catheter through the patient's vasculature to the upper left chamber of the heart (e.g., the left atrium), using a technique to go through the septum (muscular wall that divides the upper chambers of the heart into the right and left sides). A catheter may be used to place a closure device around the leak. The closure device may act like a plug to stop the leak. However, the device may be challenging to install across the leak in many cases.

Various sizes of wires may be used. An example of a larger size for intracardiac structures, e.g., ventricle and atrium, may typically be wires that are 0.035″ thick. In one example, an expanded 3D wire may be 2-4 inches once expanded. The core end may be 0.035 inches thick, standard to deliver devices. A smaller size for use within a coronary artery may typical be coronary wires that may be 0.014 thick. In an example, smaller versions may be used for support in a coronary artery. In an example, an expanded end may be 3-4 mm, connected to a standard 0.014″ core wire. The proposed device may use standard guidewire dimensions for the core wire depending on the needs of the specific case, for example 0.014 for coronary intervention, such that the proposed device may be compatible with current cardiac devices. The device may take a shape on the distal end that is larger than the guidewire thickness, and exists on a 3-dimensional plane, e.g., in X, Y, and Z planes.

In an example embodiment, a medical device may be inserted over the guidewire. Guidewires have been used to aid the introduction of medical instruments such as catheters and other medical instruments into the human body. Many medical applications and designs of guidewires have been developed for cardiovascular use. Accordingly, a device for valve leaflet modification may be inserted over the guidewire. In some example embodiments, a pre-shaped wire may be used as the guidewire. The shape may be used to make it less likely for the wire to puncture the heart, a valve within the heart, an artery, or other passage that the wire may travel within. For example, a wire may form a ball at an end of a guidewire. The spherical shape of the ball may relieve stress or spread force over a larger area of the wire. Relieving stress or spreading force over a larger area of the wire may make it less likely for the wire to puncture a sidewall of an artery or other tissue. In an example embodiment, the wire may be made from nitinol.

In an example embodiment, a guidewire may include an elongated core with a proximal end, a distal end, and a length extending along a longitudinal axis therebetween and a three-dimensional atraumatic tip disposed at the distal end of the elongated core. In an example embodiment, the three-dimensional atraumatic tip comprises a spherical shaped coil of wire. In an example embodiment, the spherical shaped coil of wire has a thickness and a diameter at least two times the thickness of the elongated core. In another example embodiment, the spherical shaped coil of wire has a thickness and a diameter at least four times the thickness of the elongated core. In an example embodiment, the spherical shaped coil of wire has a thickness and a diameter at least ten times the thickness of the elongated core. In another example embodiment, the three-dimensional atraumatic tip comprises an oblong shaped coil of wire. In an example embodiment, the three-dimensional atraumatic tip comprises an umbrella shaped coil of wire. In an example embodiment, the three-dimensional atraumatic tip comprises a three-dimensional coil of wire. An example embodiment further includes a sheath, at least one of the elongated core and the atraumatic tip disposed within the sheath prior to deployment.

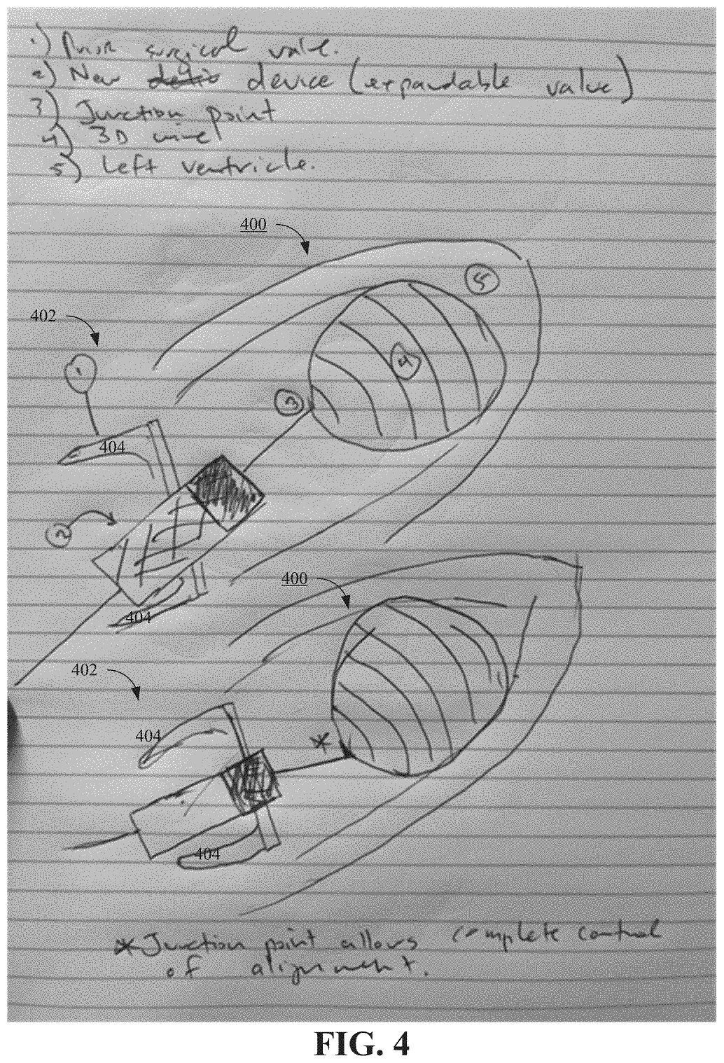

FIG. 4 is a diagram illustrating an example 3D guidewire 400 including a junction point 402. The junction point 402 may be a connection point from the 3D structure and the rest of the guidewire. An example embodiment includes a 3D shape, which may be attached to a 0.35 wire at the junction point. Another example embodiment includes a 3D shape, which may be attached to a wire at a junction point, the wire having a diameter in the range of 0.30 to 0.40. Another example embodiment includes a 3D shape, which may be attached to a wire at a junction point, the wire having a diameter in the range of 0.25 to 0.45. Another example embodiment includes a 3D shape, which may be attached to a wire at a junction point, the wire having a diameter in the range of 0.20 to 0.50.

A structure proximal from the 3D structure may allow for better control of alignment. For example, the structure may include be proximal from the junction point 402 where the 3D structure attaches to the rest of the guidewire. Additionally, the junction point 402 may include guides 404 that may slide along an artery. The fins (e.g., guides 404) may be allowed to move as needed to guide the device along the artery.

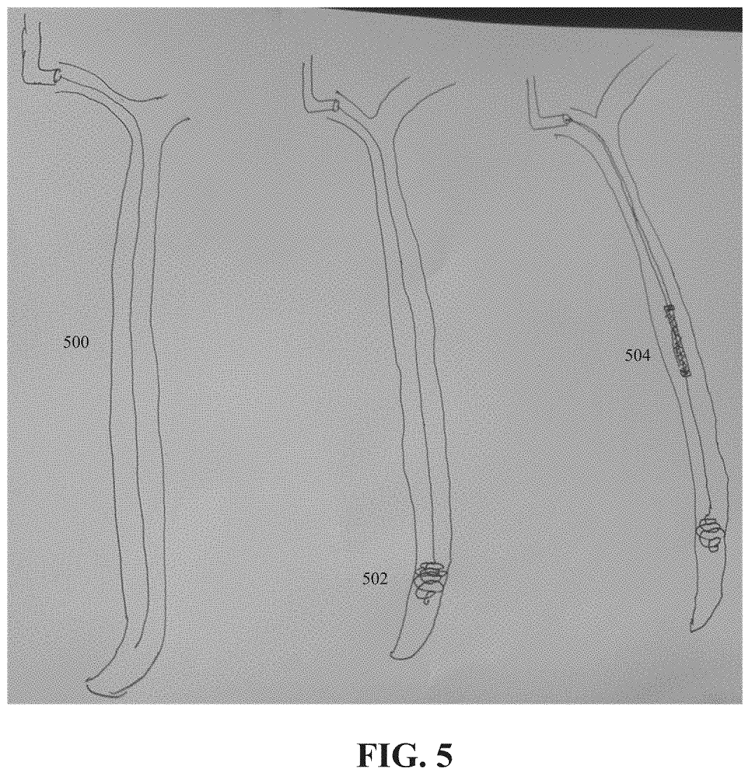

FIG. 5 is a diagram illustrating example 3D guidewires including a standard guidewire and a guidewire in accordance with the systems and methods described herein. On the left is a standard workhorse wire 500, e.g., in a coronary artery. In the middle is an example of a proposed wire 502 after an exchange. On the right is an example stent 504 being delivered. In the illustrated example, the delivery may be better, e.g., a safer delivery, using the example wire as described herein.

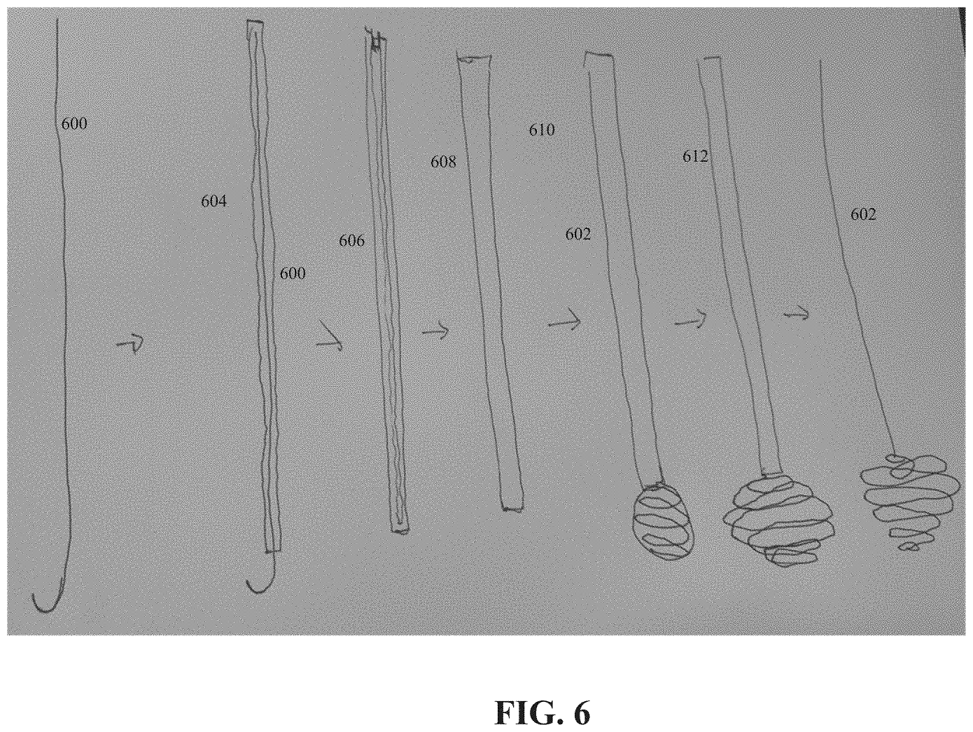

FIG. 6 is a diagram illustrating example 3D guidewires including a standard J wire 600 and a guidewire in accordance with the systems and methods described herein 602. In the illustrated example, on the left, a standard J wire 600 may be used. The standard J wire may be 0.035″ version in some examples. Next, a proposed exchange catheter 604 may be advanced over the standard J wire 600. The catheter 604 inner dilator may be advanced 606 and then removed 608. This may leave an outer sheath with a larger diameter, as the 3d component may be larger than the lumen needed for an 0.035″ wire. The proposed wire 602 could then be delivered through this sheath at 610, and take its form within the cardiac structure at 612. This example discusses a 0.035″ wire in a cardiac structure. It will be understood that different wire sizes may be used in different applications. For example, a smaller wire may be used to deliver a device into the stomach, over which imaging or other medical devices could then be safely advanced.

Next, a sheath having a size that is larger than the J wire, e.g., 0.035″, as the proposed wire may likely be bigger when delivered. As the sheath is retracted, the 3D wire may start to form. In other words, when the wire according to the systems and methods described herein emerges from the sheath, the 3D wire may begin to expand. The 3D wire may be fully formed at some point after it emerges from the sheath. In the illustrated example, the delivery sheath may be removed. Left in place is the 3D wire, e.g., with the standard 0.035″ core wire that may be used to deliver cardiac devices (e.g., a valve). The illustrated example discuses a 0.035″ core wire. It will be understood that other wire sizes may be used.

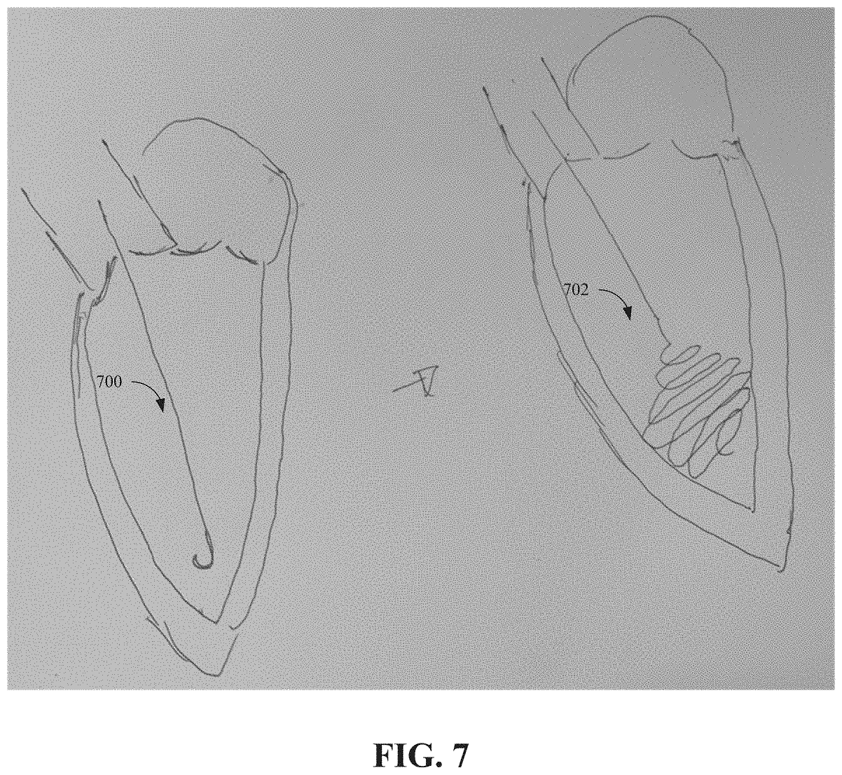

FIG. 7 is a diagram illustrating an example 3D guidewire that includes a standard J wire 700 and a guidewire in accordance with the systems and methods described herein 702. In the illustrated example, a standard wire may be advanced through an aortic valve into a left ventricle (e.g., on the left). On right, after the exchange steps, e.g., such as discussed with respect to FIG. 6, the 3D wire may be positioned as illustrated (FIG. 7, left). With the wire positioned as illustrated (FIG. 7, left), the left ventricle may be protected from puncture by the wire. The protection may be afforded by increasing force dispersion across a greater surface area.

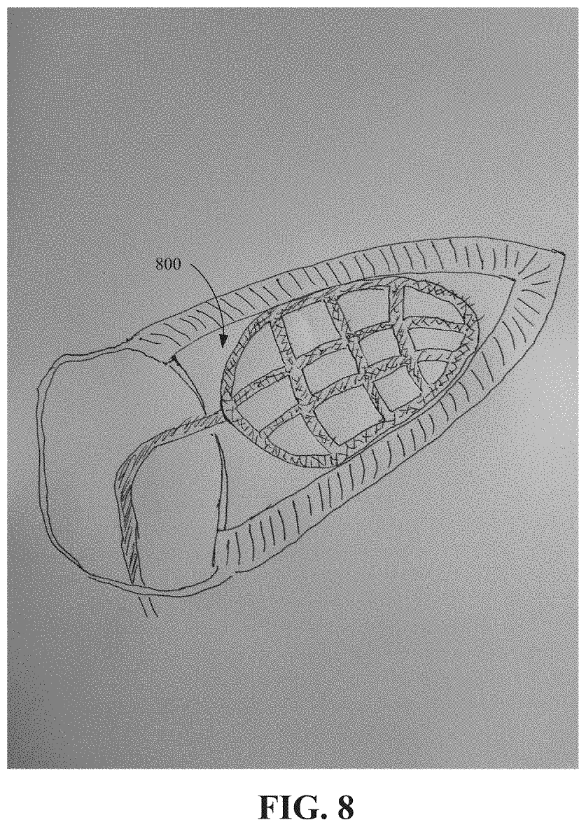

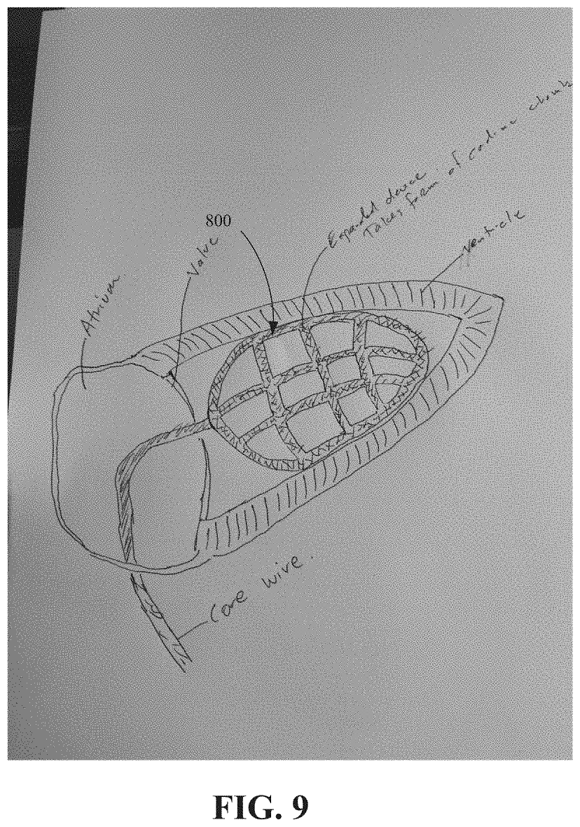

FIG. 8 is a diagram illustrating an example device 800 when expanded in accordance with the systems and methods described herein. FIG. 9 is also diagram illustrating the example device 800when expanded in accordance with the systems and methods described herein.

An example may be self-expanded (e.g., made from nitinol). An example may be a device that may be balloon expandable. For example, an aspect may use a smaller 0.014″ wire with a balloon that encompasses a multidimensional wire. Some embodiments may use a wire that may be between 50 microns and 5 mm. Another example would be a core wire attached to a distal end that is fully balloon expandable. In an example, once in cardiac chamber, a balloon may be expanded. Accordingly, the device may take a 3D shape, which may conform to the cardiac chamber. This would provide stability to the core wire during device delivery and protect surrounding structures. The image demonstrates a fully expanded depiction of the 3D component within the cardiac chamber, here left ventricle, with balloon expandable configuration. The image shows a balloon expandable version that may be fully expanded and 3-dimensional. Note that the balloon may be filled with air, liquid, contrast media or some combination of materials.

An example embodiment may have radiopaque markers. An example embodiment may have electrical conduction material around balloon to sense activity. FIGS. 8-9 illustrate an example device when expanded, e.g., in the cardiac chamber. In full 3-dimensional form, the device protects surrounding structures and conforms to the native anatomy.

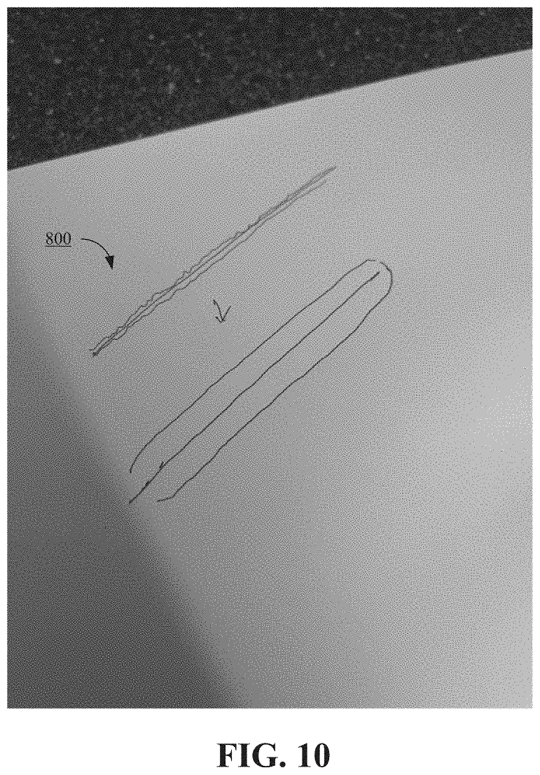

FIG. 10 is a diagram illustrating an example side view of the device 800 including a small wire inside and outer balloon that may expand the device when the balloon is expanded in accordance with the systems and methods described here. The first example illustrates a side view of a small wire with outer balloon that has not been expanded. The small wire may transition to a state with an outer balloon that is expanded. This would be the appearance from a side view throughout the 3d structure once expanded. In various embodiments, the device may be designed as a balloon expandable structure with or without a wire inside.

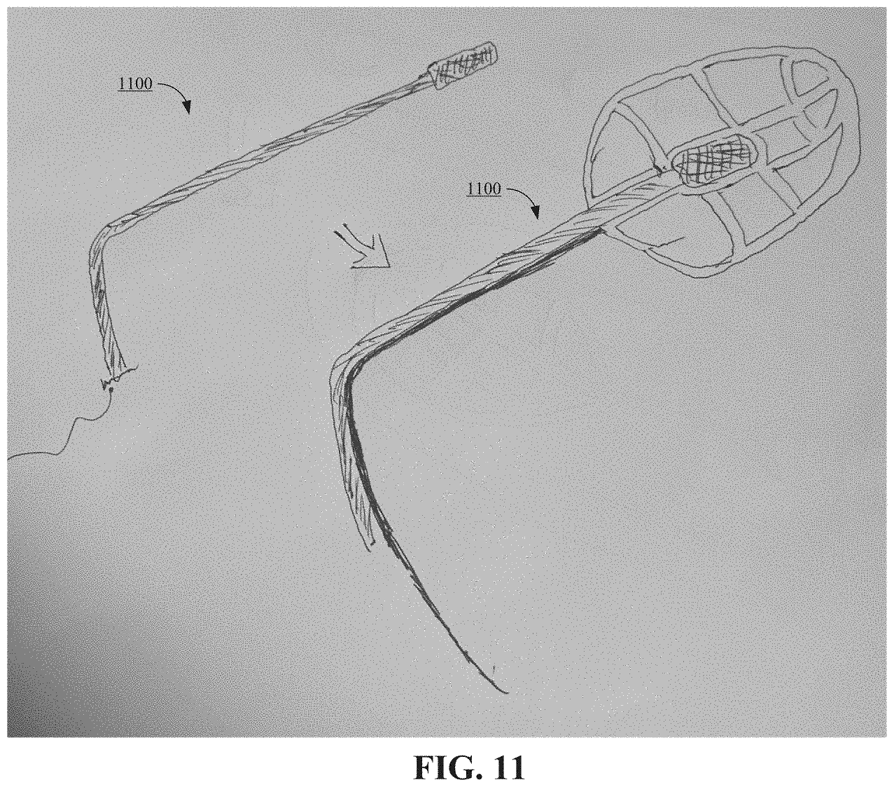

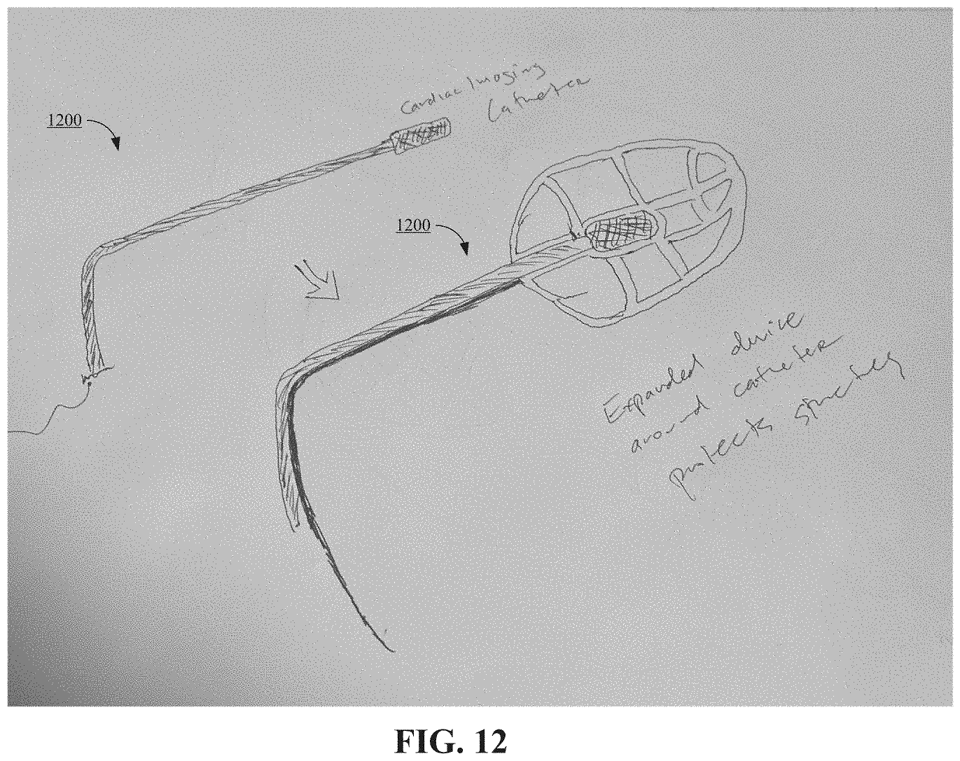

FIG. 11 is a diagram illustrating an example device 1100 that may be placed around an existing cardiac device and inserted into a body in accordance with the systems and methods described herein. FIG. 12 is a diagram illustrating an example device 1200 that may be placed around an existing guidewire and inserted into a body in accordance with the systems and methods described herein.

In an example, a 3D device may be placed around an existing device, e.g., a cardiac imagine catheter, and using the same principles and design, protect surrounding body structures. FIGS. 11 and 12 show a cardiac imaging device that may be directly inserted from the femoral artery into the right atrium or left atrium. There have been cases described where the cardiac imaging device caused damage and perforation of the heart, which can be deadly. Specific examples include, but are not limited to intracardiac echo (ICE), large cannulas for used to circulate blood on a heart-lung machine, imaging devices advanced into the esophagus (i.e. transesophageal echocardiography) for imaging the heart, imaging devices advanced through the esophagus to image hepatic, pancreas, stomach (endoscopic ultrasound). The proposed device could be placed around an existing device as a sleeve. Once in the appropriate position, this outer sleeve could be inflated from outside the body to take a 3-dimensional form and protect surrounding structures. In FIG. 12, we demonstrate this concept with an intracardiac echocardiography (ICE) device. After the device is inserted into the correct chamber, the outer sleeve protective structure would take form to protect surrounding structures. Note that within a cardiac chamber, this device would have open cells to allow free flow of blood once the device is inflated. These open cells allow the heart to function normally while the device is deployed in 3-dimensional form.

Generally, blood needs to flow freely during procedure. Another embodiment might be used to protect the esophagus from transesophageal echocardiography (TEE) probe.

In this embodiment, again would be placed around TEE probe and advanced into esophagus. There is no blood in esophagus, in such an example, the proposed device may be fully closed without open cells. In this embodiment, the device could be filled with fluid to enhance imaging. Ultrasound is dependent on direct contact with body tissue or fluid filled medium. Air, which may be present in the esophagus, presents optimal imaging. The proposed device would remove any potential for air between the structure being imaged and thus improve imaging while also protecting the surrounding structure (in this case the esophagus).

An example device may also be added to a preexisting device, e.g., a replacement valve.

In an example, the proposed 3D device may be placed in front of another device on same wire, e.g., a J wire, separated. In an example, the TAVR valve may push behind proposed device.

In an example, a 3D device may be added to a pre-existing device by a manufacturer, such that the 3D device may be directly attached to the device. In an example embodiment a 3D device may be added to a pre-existing device as an outer covering, e.g., an ICE catheter. ICE catheters are advanced directly through a vessel into a cardiac chamber, without the use of a guidewire. The 3D device may be placed on the outside of an existing device of the device as a sleeve. Once in a heart chamber, the 3-dimensional shape of the 3D device may then be deployed, e.g., via balloon inflation as proposed earlier to protect cardiac catheters. The device could also be added to the end of an existing device, e.g., transcatheter valve. The device may be added to the end of an existing device to improve safety of the device.

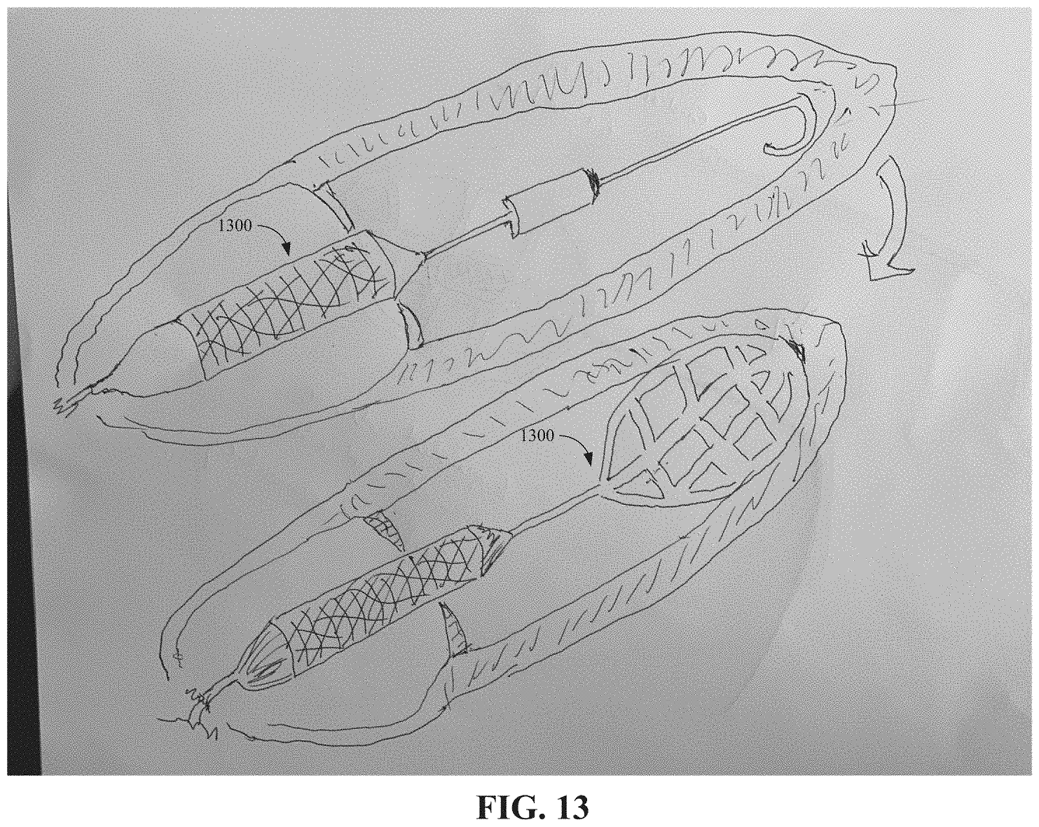

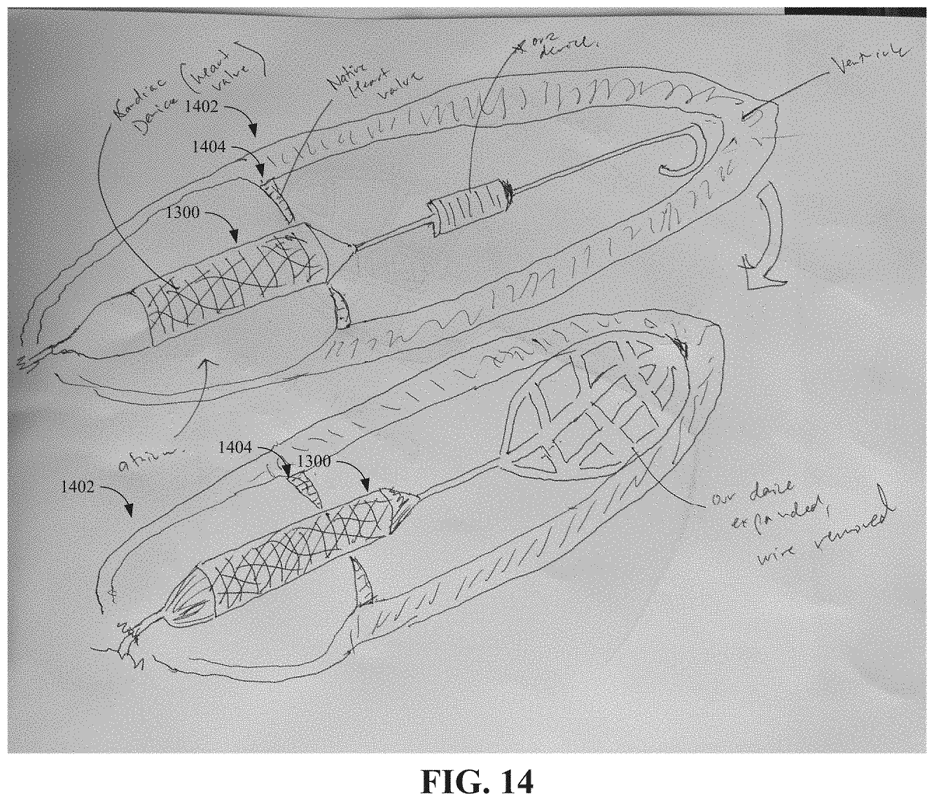

FIG. 13 is a diagram illustrating an example device 1300 inserted into a body in accordance with the systems and methods described herein. FIG. 14 is a diagram illustrating the example device inserted into a body in accordance with the systems and methods described herein. The example of FIGS. 13-14 illustrate an example 3D device 1300 in a heart 1402 and entering through a heart value 1404.

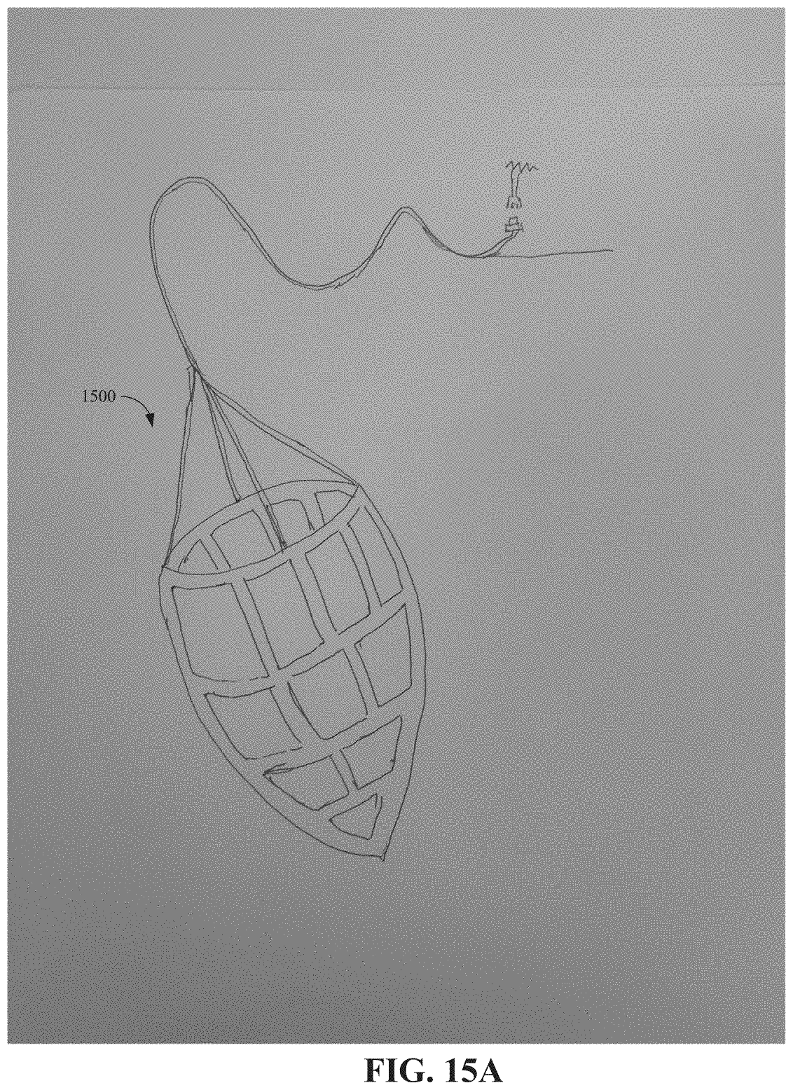

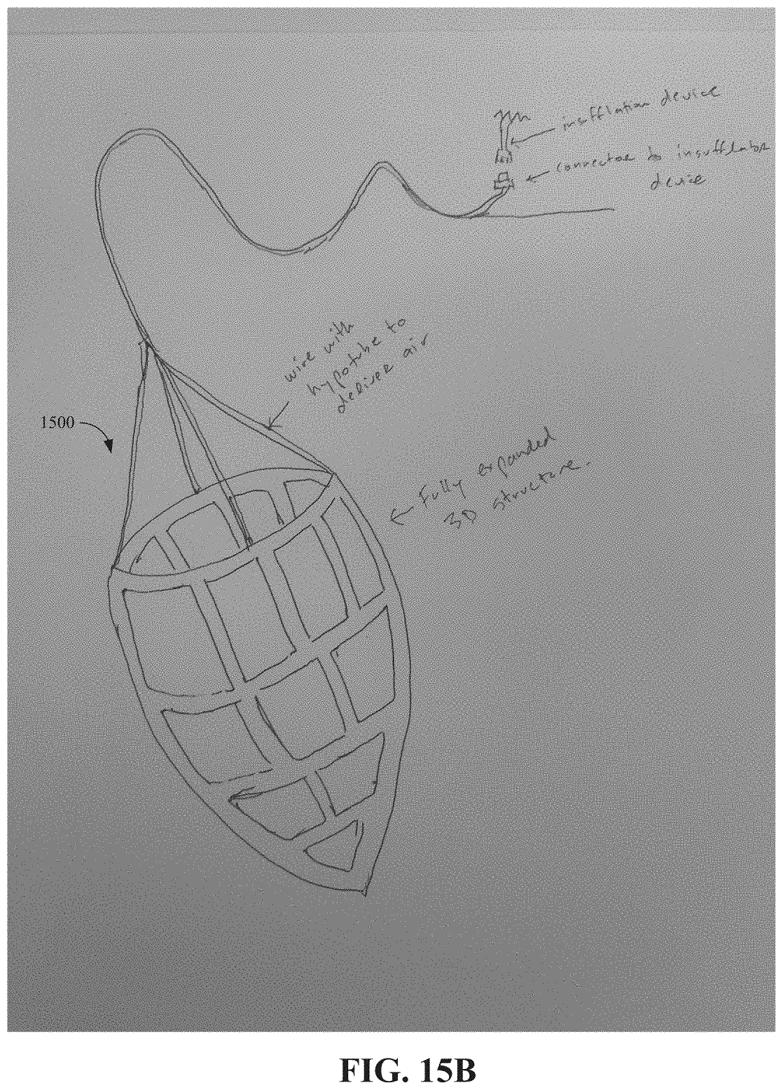

FIGS. 15A-15B are diagrams demonstrating a 3-dimensional view of an example embodiment of the proposed device 1500. This version describes an expandable version that uses air or liquid to fill the device. Also illustrated is a side port, also known as a hypotube, that may be used to deliver liquid or air into the device to expand into 3-dimensional shape. This version may have open cells to allow blood to flow through the device. The device may be constructed as a multidimensional balloon that takes a 3-dimensional form. The device may also be constructed as a multidimensional balloon with attached wire to provide additional support.

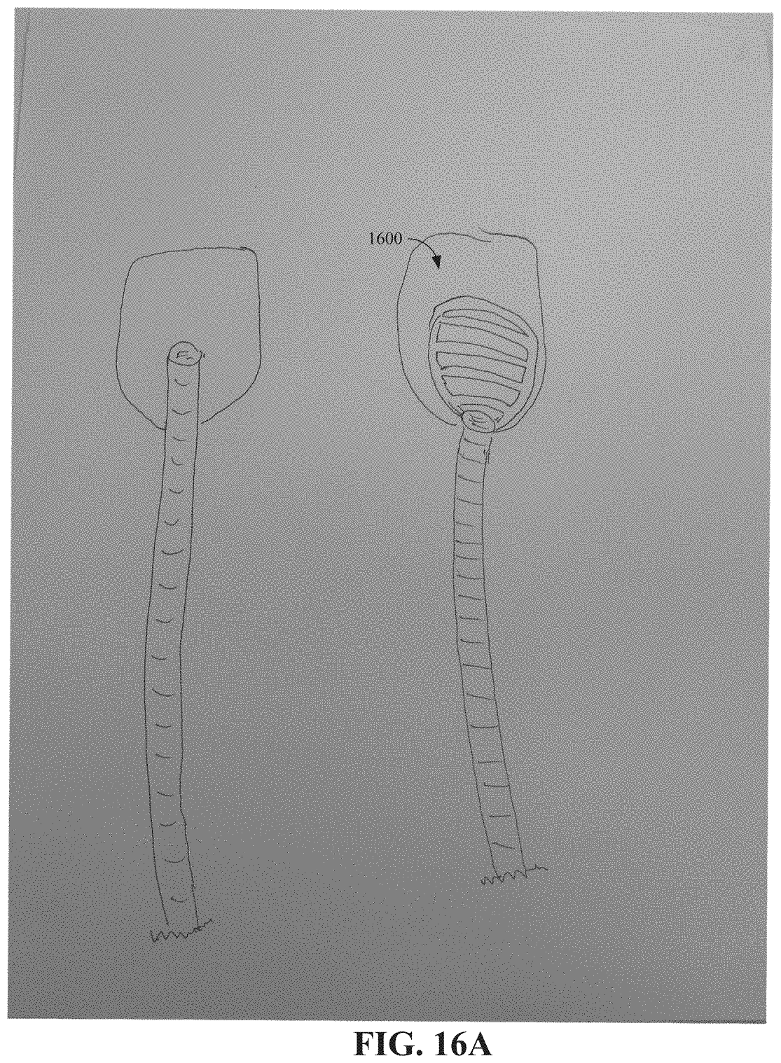

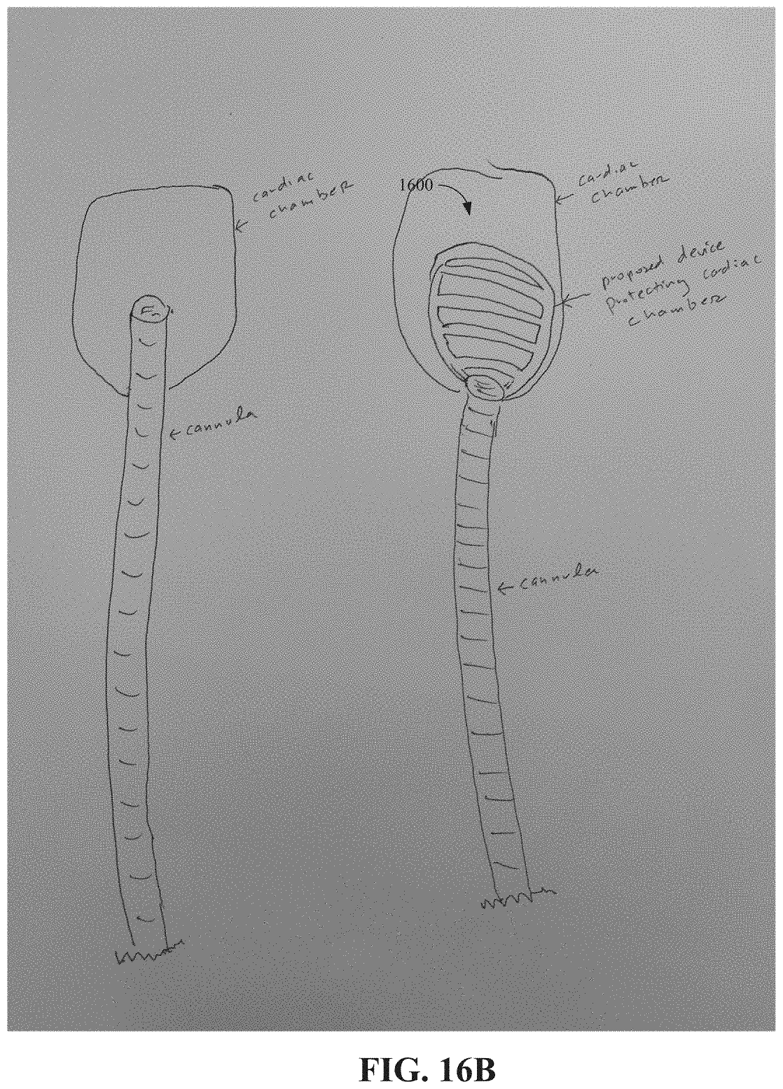

FIGS. 16A-16B are diagrams demonstrating use of an example device 1600 on a cannula. Cannulas are large hollow tubes that are inserted from outside the body and into the body. Cannulas may commonly be attached to a heart bypass machine, (also known as extra corporeal membrane oxygenation, bypass) to pump blood through the heart during procedures or in cases of shock or hypoxia. The cannulas have been known to perforate the chamber in which they reside, which can be deadly. FIGS. 16A-16B illustrate a typical cannula resting in a cardiac chamber on the left. On the right side, the example proposed device is demonstrated. As can be seen, the device may take a 3-dimensional shape that may protect the surrounding chamber. This may be open celled, allowing blood to flow around the device. The example device may be expanded through the same techniques described herein. The device may be manufactured in a way such that the example device may be attached directly to the end of a cannula. The example device may also be advanced over an existing cannula as a sleeve.

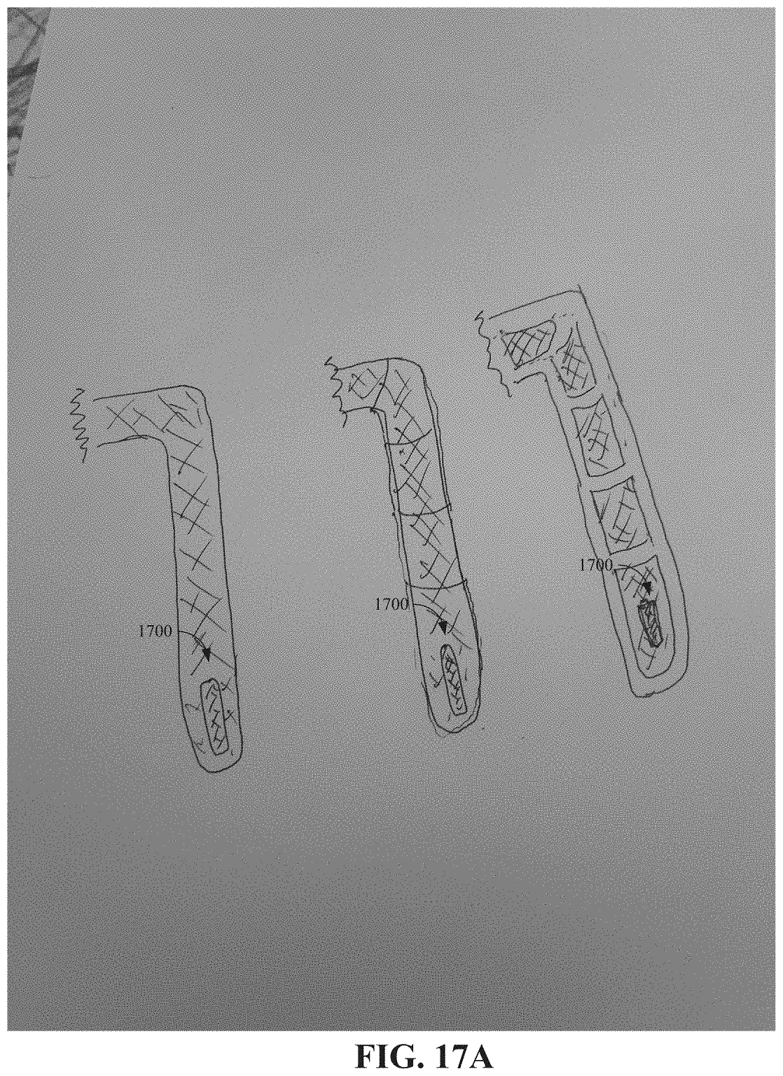

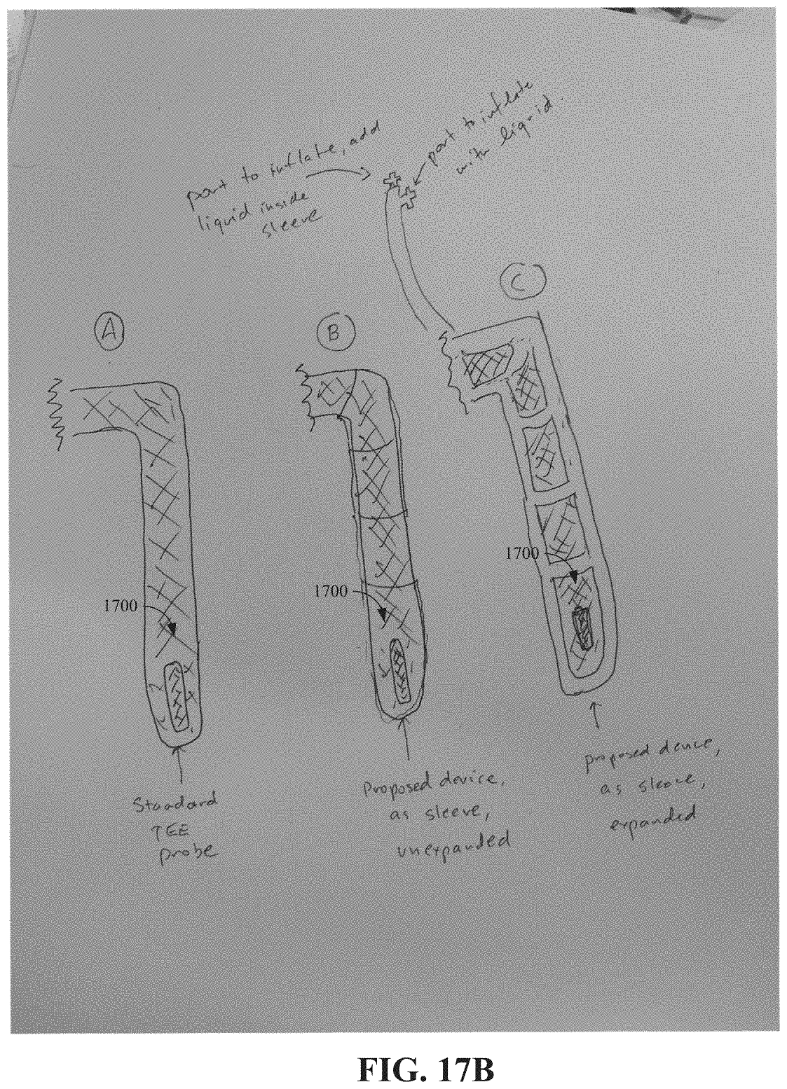

FIGS. 17A-17B are diagrams demonstrating use of an example device 1700 with transesophageal echocardiography (TEE). A TEE probe is advanced through the esophagus and used to image cardiac structures. The device is advanced directly into the esophagus, and has been reported to cause esophageal damage including perforation. Part A on the left demonstrates a standard TEE probe, which can be imagined here in the esophagus. Part B shows an example of the proposed device used as a sleeve around the TEE probe. In Part B, the sleeve is unexpanded. Part C illustrates the device in full expanded view. Note that the device may have 2 ports. The first port may be used to expand the device with a liquid. The expansion of the device would protect the esophagus from damage by the TEE probe and manipulation of the probe. The second port may be used to fill the sleeve itself with a liquid. Ultrasound cannot travel through air, and a TEE may be dependent on constant contact with the esophagus. By filling the sleeve with liquid, imaging quality would be improved as there would be no air to obstruct ultrasound imaging.

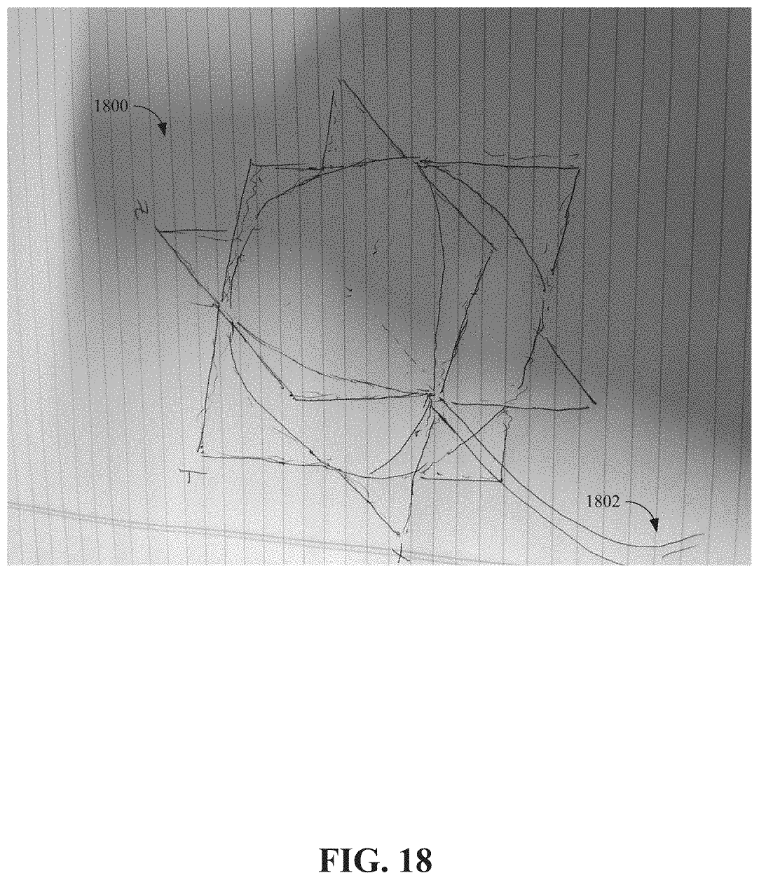

FIG. 18 is a diagram illustrating how various devices 1800, such as cardiac devices may advanced into the body over standard sized guidewires 1802. For example, as described above, a transcatheter aortic valve may be advanced over a 0.035 inch guidewire. It will be understood that other sizes of guidewire may be used for other purposes in conjunction with one of more other aspects of the systems and methods described herein. The wires may be advanced through a catheter that has an inner diameter just larger than the standard guidewire. As the wire is advanced through the delivery catheter, the wire may take a shape along an X and Y axis as illustrated in FIG. 18. The proposed device may be designed such that the proposed device may be delivered through a standard catheter, but take a geometric spherical form along an X, Y, and Z axis.

Various examples are described herein, such as devices that may be used in the heart, the esophagus, or other bodily openings. It will be understood that the systems and methods described herein may be used with respect to other bodily openings in order to protect tissues reached through those bodily openings.

The figures and the description above describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The foregoing description of the embodiments of the present invention has been presented for the purposes of illustration and description. It is not intended to be exhaustive or to limit the present invention to the precise form disclosed. Many modifications and variations are possible in light of the above teaching. It is intended that the scope of the present invention be limited not by this detailed description, but rather by the claims of this application. As will be understood by those familiar with the art, the present invention may be embodied in other specific forms without departing from the spirit or essential characteristics thereof. Likewise, the particular naming and division of the modules, routines, features, attributes, methodologies and other aspects are not mandatory or significant, and the mechanisms that implement the present invention or its features may have different names, divisions and/or formats.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

What is claimed is:

1. A guidewire comprising:
an elongated core with a proximal end, a distal end, and a length extending along a longitudinal axis therebetween; and
a three-dimensional atraumatic tip disposed at the distal end of the elongated core.

2. The guidewire of claim 1, wherein the three-dimensional atraumatic tip comprises a spherical shaped coil of wire.

3. The guidewire of claim 2, wherein the spherical shaped coil of wire has a diameter and a diameter at least two times the thickness of the elongated core.

4. The guidewire of claim 2, wherein the spherical shaped coil of wire has a thickness and a diameter at least four times the thickness of the elongated core.

5. The guidewire of claim 2, wherein the spherical shaped coil of wire has a thickness and a diameter at least ten times the thickness of the elongated core.

6. The guidewire of claim 1, wherein the three-dimensional atraumatic tip comprises an oblong shaped coil of wire.

7. The guidewire of claim 1, wherein the three-dimensional atraumatic tip comprises an umbrella shaped coil of wire.

8. The guidewire of claim 1, wherein the three-dimensional atraumatic tip comprises a three-dimensional coil of wire.

9. The guidewire of claim 2, further comprising a sheath, at least one of the elongated core and the atraumatic tip disposed within the sheath prior to deployment.

10. A method of a guidewire, the method comprising:
providing the guidewire, the guidewire comprising an elongated core with a proximal end, a distal end, and a length extending along a longitudinal axis therebetween and a three-dimensional atraumatic tip disposed at the distal end of the elongated core, at least one of the elongated core and the atraumatic tip disposed within a sheath prior to deployment the elongated core and the atraumatic tip; and
deploying the elongated core and the atraumatic tip.

11. The method of claim 10, wherein the three-dimensional atraumatic tip comprises a spherical shaped coil of wire.

12. The method of claim 10, wherein the three-dimensional atraumatic tip comprises an oblong shaped coil of wire.

13. The method of claim 10, wherein the three-dimensional atraumatic tip comprises an umbrella shaped coil of wire.

14. The method of claim 10, further comprising retracting the elongated core and the atraumatic tip.

15. A 3-D protective structure comprising:
a three-dimensional atraumatic structure, the three-dimensional atraumatic structure configured to be attached to a medical device at a distal end of the medical device to protect a patient from having the medical device cause trauma to a structure within the body of the patient.
